(19) United States
(12) Patent Application Publication
(10) Pub. No.: US 2023/0407186 A1
(43) Pub. Date: Dec. 21, 2023

(54) ELECTRIC FURNACE TO PRODUCE OLEFINS

(71) Applicant: LUMMUS TECHNOLOGY LLC, Houston, TX (US)

(72) Inventors: Kandasamy Meenakshi Sundaram, Houston, TX (US); Stephen J. Stanley, Houston, TX (US)

(73) Assignee: LUMMUS TECHNOLOGY LLC, Houston, TX (US)

(21) Appl. No.: 18/251,361

(22) PCT Filed: Nov. 2, 2021
(86) PCT No.: PCT/US2021/057700

§ 371 Date: May 1, 2023

Related U.S. Application Data
(60) Provisional application No. 63/108,699, filed on Nov. 2, 2020.

(51) Int. Cl. C10G 9/24

(52) U.S. Cl. CPC C10G 9/24; C10G 2300/4037

(57) ABSTRACT

A method of thermally cracking a hydrocarbon feed (105) includes feeding the hydrocarbon feed (105) into at least one coil (130) in a reaction section (112) of an electric heater (110), using electrical energy to heat the hydrocarbon feed (105) in the electric heater (110) to a reaction temperature, and directing a reaction output from the electric heater (110) to at least one exchanger (150) to cool the reaction output.

### BACKGROUND

Furnaces used in pyrolysis are generally fired heaters, which use hot combustion gases (flue gases) or gaseous and liquid fuels to generate heat and supply the reaction duty. The heat raises the temperature of fluid flowing through the coils arranged inside the fired heater. Thermal cracking reactions take place in the radiant section of the fired heater. These are highly endothermic reactions and heat is added in order to maintain the reaction. Typically, 30% to 50% of the fired duty is used to carry out the reaction in the radiant section of the heater. The remaining duty in the flue gas is recovered in the convection section of the heater and may be used for preheating the feed and/or steam generation.

### SUMMARY OF THE DISCLOSURE

In one aspect, embodiments of the present disclosure relate to reactors for cracking a hydrocarbon feed that include a heater chamber defining a reaction section of an electric heater, a plurality of electrical heating elements disposed around the heater chamber, wherein the electrical heating elements are electrically powered, at least one coil extending from a feed inlet through the reaction section, and a primary exchanger having an inlet fluidly connected to the at least one coil and an effluent outlet.

In another aspect, embodiments of the present disclosure relate to methods of thermally cracking a hydrocarbon feed that include feeding the hydrocarbon feed into at least one coil in a reaction section of an electric heater, using electrical energy to heat the hydrocarbon feed in the electric heater to a reaction temperature, and directing a reaction output from the electric heater to at least one exchanger to cool the reaction output.

In yet another aspect, embodiments of the present disclosure relate to methods of designing a thermal cracking plant that include an electric heater for thermally cracking a feed and a recovery section, determining an amount of steam generated and an amount of steam consumed by the thermal cracking plant, determining an amount of power used by the thermal cracking plant to thermally crack the feed, and adjusting at least one parameter of the thermal cracking plant to reduce the amount of power used by the thermal cracking plant.

The diagrams shown in the attached sketches can be slightly modified for specific crudes and hydrocarbon feedstocks and product slates. Other aspects and advantages will be apparent from the following description and the appended claims.

### BRIEF DESCRIPTION OF DRAWINGS

FIG. 1 is a diagram of an electric heater according to embodiments of the present disclosure.

FIG. 2 is a simplified process flow diagram of a system for cracking hydrocarbon mixtures according to embodiments herein.

FIG. 3 is a graph of expected ethylene yield and coil outlet temperature (COT) as a function of residence time.

FIG. 4 shows a graph comparing the metal temperature of coil metal when heated by a fired heater and when heated by an electric heater.

### DETAILED DESCRIPTION

Embodiments disclosed herein relate generally to the cracking of hydrocarbons to produce light olefins, such as ethylene, propylene, etc. using electric heaters to heat the hydrocarbon feeds to a reaction temperature. Electric heaters may also be referred to as electric furnaces. Hydrocarbon feeds useful in embodiments herein may range from light hydrocarbons (ethane, propane, butanes) and naphtha range hydrocarbons (C5s to C12s) on up to heavier hydrocarbon gas and mixtures thereof, including whole crudes.

Thermal cracking of hydrocarbons is commonly used to produce light olefins. For example, when ethane is cracked, it produces mainly ethylene. When naphtha is cracked, it may produce ethylene, propylene, butenes, butadiene, and benzene as valuable products. Thermal cracking reactions are highly endothermic, where heat is supplied to sustain the reaction. To get appreciable feed conversion, the reactor temperature may be well above 700° C., e.g., greater than 800° C.

In some cracking processes, catalysts can be employed to reduce the operating temperature, but may result in less ethylene yield than thermal cracking. Though the heat of reaction is nearly the same for thermal and catalytic cracking per unit weight of olefins produced, fired duty for thermal cracking is extremely high. To sufficiently heat the feed (e.g., to high temperatures greater than 800° C.) for ethylene production, a higher proportion of sensible heat (energy required to change the temperature of a substance with no phase change) to the reaction duty may be used. Sensible heat can be recuperated by exchanging with other process fluids, and thus ethylene heaters may be designed to efficiently preheat the feed and to generate additional steam. When using electric heaters according to the present disclosure, since there is no flue gas containing high thermal energy, either the electric heater may be designed to preheat the feed and to carry out the reaction or other more efficient ways of preheating the feed may be used.

Cracking reactions may produce a small amount of coke as a byproduct, which may deposit and build up in the reactor. To minimize coke deposition and to improve the olefin production, steam may be added to the hydrocarbon feed and cracked.

In fired heaters, feed mixture (hydrocarbon and dilution steam (DS)) is usually preheated in the convection section of the fired heater and enters the radiant section of the heater where the reactions take place. Since these are high temperature reactions, high temperature flue gas is generated from the reaction in the fired heaters. Typically, only 30 to 50% of the fired duty from fired heaters goes to the reaction section, where the remaining amount of the fired duty may leave the radiant section as flue gas. The energy in the flue gas may be recovered in the convection section of the fired heater, which may include coils arranged suitably therein to recover the heat from the flue gas. In the convection section of fired heaters, the feed and the dilution steam may be preheated and also superheated to a desired temperature before entering the radiant section. Even after heating the feed mixture surplus, thermal energy is present in the flue gas. If this energy is not recovered, then the energy is wasted and the cost of olefin production goes up. In contrast, when using electric heaters according to embodiments of the present disclosure, 90 to 98% of the electrical energy used by the electric heater may go to the reaction in the in the reaction section of the heater. Thus, electric heaters disclosed herein may generate only enough energy sufficient for the reaction, where little to no excess heat is generated. Without a significant amount of excess heat being generated, electric heaters disclosed herein may have no convection section.

To preserve olefins formed in the reactor, the reaction output (also referred to as effluent) may be quenched quickly. Old methods of quenching used injection of oil or water at the reactor outlet. More recent quenching methods have used indirect cooling. In some methods, effluents may be cooled by generating high pressure (or super high pressure) steam before sending effluents to a recovery section. This high pressure steam was traditionally superheated in the convection section of a fired heater. However, when using electric heaters according to embodiments of the present disclosure that do not have a convection section, steam may be generated in other parts of the process (e.g., in a recovery section where effluents are cooled, such as in exchangers, or using a secondary electric heater).

According to embodiments of the present disclosure, a reactor for cracking a hydrocarbon feed may include an electric heater and at least one exchanger, which may be used to cool reaction output from the electric heater and/or preheat feed going into the electric heater. An electric heater may include a heater chamber defining a reaction section of the heater, a plurality of electrical heating elements disposed around the heater chamber, wherein the electrical heating elements are electrically powered, and a plurality of coils extending from a feed inlet of the reaction section to an outlet of the reaction section. In some embodiments, a primary exchanger may be used to initially cool the reaction output from the electric heater, where the primary exchanger may have an inlet fluidly connected to the plurality of coils and an effluent outlet. In some embodiments, a secondary exchanger may be used to further cool the primary exchanger effluent, where the secondary exchanger may have an inlet fluidly connected to the effluent outlet of the primary exchanger. In some embodiments, a tertiary exchanger may be used to further cool the secondary exchanger effluent, where the tertiary exchanger may have an inlet fluidly connected to the effluent outlet of the secondary exchanger.

Exchangers may further include steam outlets and/or steam flow line(s) which may direct heated steam to one or more areas of the reactor and/or to a preheating section. For example, heated steam from an exchanger may be directed toward the feed inlet of an electric heater to preheat feed prior to entering the electric heater. A preheating section may be provided separately from the reaction section of an electric heater or may be provided with the reaction section as a single unit. For example, a preheating section of a reactor may be spaced apart from the reaction section and downstream of the feed inlet of the electric heater. In some embodiments, the preheating section may include one or more exchanger. A feed inlet to an electric heater may be fluidly connected to multiple feed sources.

According to embodiments of the present disclosure, a main reaction section of an electric heater may have different arrangements of one or more coils extending through the reaction section of the electric heater. The coils may be heated by different heating elements in a single electric heater, or coils in the reaction section may be heated with a single heating element in the electric heater. Both preheat and reaction heat may be supplied by the single electric heater.

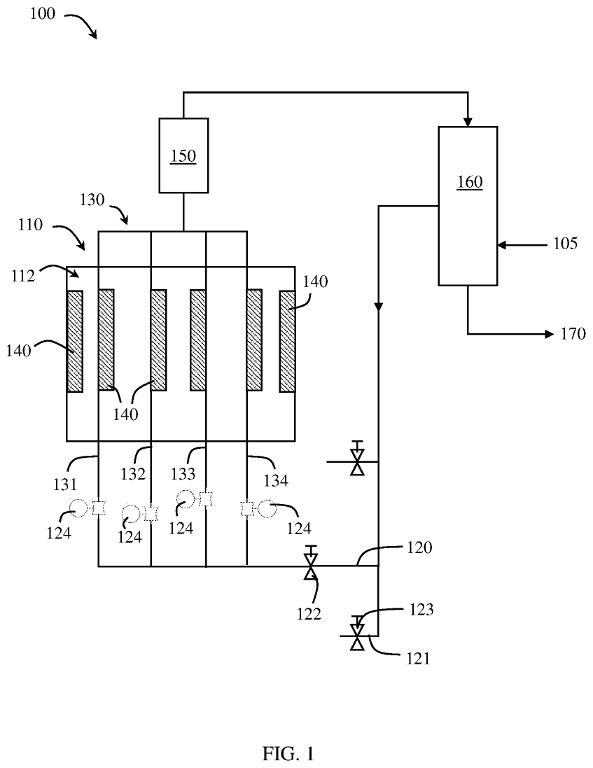

FIG. 1 shows an example of a reactor 100 using an electric heater 110 according to embodiments of the present disclosure. The electric heater 110 provides the main reaction section of the reactor, where a hydrocarbon feed 105 may be heated to a reaction temperature to crack the hydrocarbon feed. The hydrocarbon feed 105 may be heated through a secondary exchanger 160 and flowed through flowline 120 to one or more coils 130 extending through a reaction section 112 of the electric heater 110. The reactor 100 may not include a convection section (as found in fired heaters), but instead may include flowlines 120 fluidly connected to coils 130 disposed in the electric heater 110 (for feeding one or more feeds to the electric heater) and one or more electrical heating elements 140 disposed around the coils 130 in the electric heater 110. The reactor 100 may further include feed exchangers (e.g., primary exchanger 150 and secondary exchanger 160) and common flowlines (e.g., through headers) from the feed exchangers feeding various coils in the reaction section 112 of the reactor 100. Thus, in contrast to fired heaters, the electric heater 110 may not include a convection section. Instead, feed exchangers and common flowlines (e.g., headers) may direct feed to the coils 130.

Reactors using an electric heater 110 according to embodiments of the present disclosure may utilize a coil concept to crack a feed running through the coil(s) 130. In the embodiment shown, four radiant coils 131, 132, 133, 134 (collectively referred to as 130) may be arranged in the electric heater 110 to extend through the reaction section 112 of the electric heater 110. However, more or less than four coils may be arranged to extend through the reaction section of the electric heater 110. The reaction section 112 of the electric heater 110 may have one or more electrical heating elements 140 positioned around a wall forming the reaction chamber of the electric heater 110, where the heating elements 140 may be directed to heat the reaction section 112. When a feed is flowed through the coils 130, electrical heating elements 140 around the coils 130 may be used to heat the feed flowing through the coils 130 to the cracking reaction temperature.

According to embodiments of the present disclosure, each coil 130 may be independently controlled, including the amount, if any, of feed flowing through the coil and the temperature of the coil. For example, if a radiant coil 130 is connected to different feed manifolds, that coil 130 can crack the fluidly connected feeds as each feed flows through the coil(s) 130. By providing a reaction section of a reactor 100 that may accept multiple feeds, equipment for a cracking process may be compacted (e.g., rather than using multiple heaters for multiple feeds, multiple feeds may be directed to a single electric heater 110), which may save plot space in an overall plant design.

The quantity of feed to the coils 130 may be controlled via a control valve 122. In embodiments where two or more different feeds are fluidly connected to a coil 130, a control valve 122 positioned along the flow line 120 from the feed source to the coil 130 may be controlled to allow an amount of a feed to flow through the coil 130. Further, a flow venturi 124 may be associated with each coil to provide flow rate control of feed flowing into the coils 130. When flowing through the coil(s) 130, the feed may be heated to a reaction temperature to crack the feed using electrically provided heat from the electrical heating elements 140 in the electric heater 110. For example, the same coil (e.g., 131, 132, 133, or 134) provided in an electric heater 110 according to embodiments of the present disclosure may be used to crack ethane in one run, crack naphtha in another run, and in another instance, the coil can be in decoke mode. Thus, by using a coil concept, where feed is flowed through coils positioned inside the reaction section 112 of the reactor 100 to crack the feed, specific processing conditions for each coil may be controlled to crack whichever feed is flowing through the coil.

One or more additional flow lines 121 and valves 123 (e.g., an isolation valve or gate valve) may be fluidly connected to flow line 120 and used to direct steam or a steam and air mixture through the coils 130 for decoking of the radiant coils (periodically removing coke buildup on interior surface of radiant tubes). For decoking purposes, components in an electric heaters may be arranged similar to like components in a conventional fired heater, with the exception being that instead of using flamed heating, the electric heaters may use one or more electrical heating elements. By arranging components such as coils in an electric heater in a similar manner to like components in a fired heater, transfer line valves can be installed to isolate cracker effluents from decoking effluents. Further, high temperature isolation valves may be used for simpler decoking procedures (e.g., where isolation valves may be used to isolate one or more coils for decoking). When high temperature isolation valves are not used, the effluents may be cooled sufficiently to where the coil and the exchanger(s) may be decoked only by steam. When steam or air is used for decoking, a high temperature isolation valve may be used to divert effluents to a decoke drum. Decoking effluents may also be directed with cracker effluents to a recovery section of the reactor.

The electric heater 100 may include one or more heating elements 140 distributed around the coil(s) 130, such that the electrical heating may be evenly distributed around the coils 130 in the reaction section 112. In contrast to the electric heaters 110, burners in fired heaters liberate intense heat in a small volume (flame shape). Thus, in fired heaters, the coil surface facing the burner may reach very high temperatures while the coil surface perpendicular to the burner may reach a relatively very low temperature at a given length of the heater. The temperature gradient formed by directional radiation of heat in fired heaters from a flame may sometimes be referred to as the shadow effect. Because of the shadow effect, the peak temperature in a fired heater may be different than the average temperature. In such manner, the fired heater tube design may be dictated by the peak temperature. For example, firebricks used to form fired heaters are designed to withstand the higher peak temperatures in the heater. Additionally, since heat from the flame is transferred by conduction, conductivity is designed to be high to transfer heat faster.

In electric heaters of the present disclosure, electrical heating may be controlled at a constant heat flux and directed to all sides of a coil (e.g., around the entire circumference of the coil). Additionally, while it is difficult to control the heat input to every section of a coil (e.g., bottom 20% of the coil or top 20% of the coil) for a fired heater, electrical heating according to embodiments of the present disclosure may include segmenting the heater to have heating elements heat up multiple different sections of a coil, such that the whole tube may be heated uniformly. In some embodiments, a control system may be used to control the temperature of individual coils and/or individual segments of individual coils to provide a particular heating profile of a coil for a particular cracking process. By using electric heaters according to embodiments of the present disclosure, a more controlled and uniformed heating profile may be provided to the coils in the heater, which may improve the heat transfer performance significantly, reduce the peak tube temperature, and improve the selectivity to olefins.

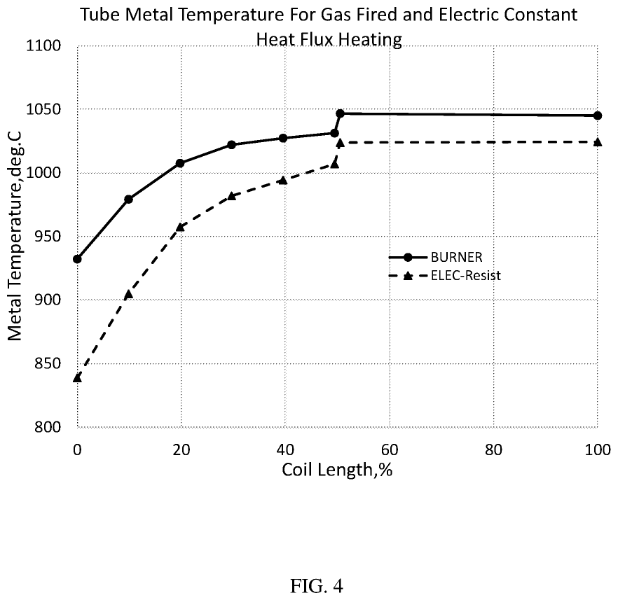

FIG. 4 shows a graph comparison of heating performance for a coil metal temperature when heated by a fired heater (from a burner) and when heated by a constant heat flux from an electric heater. As shown in FIG. 4, when using electrical heating, the radial temperature gradient may be minimized (since there is no peak temperature to average temperature difference), and thus, lower heating temperatures may be used to reach a desired metal temperature.

Further, the amount of heat to a single coil or a group coils can be individually controlled in an electric heater 110 since the heat may be supplied by individual heating elements 140. In a conventional fired heater, the whole firebox gets heated from the burners. Adjusting one or more burners directed to a single coil influences the adjacent coil heat distribution unless each coil is housed in a separate cell. With electrical heating, heating and insulation can be segregated without affecting other coils. Therefore, when an electric heater has many coils, each coil may be independently controlled. Further, heat input along different sections of a coil may be controlled. For example, high heat flux at an inlet section of the coil and low heat flux toward an end of a coil may be achieved by adjusting heater parameters of one or more electrical heating elements. By varying the heat profile along a coil, the reaction in the coil can be controlled and/or the coking rate may be controlled. Depending upon the furnace design, temperature and/or flux distribution may be imposed. Based on the performance of independently controlled coils in the cracking process, the temperature control of individual coils may be optimized to improve the coil's performance.

With an electrical heater, the heat load can be varied from 0 to 100% and hence turndown or adjusting the severity of heat (or coil outlet temperature (COT)) may not be an issue. With a fired heater, very low turndown is not possible due to the probability of extinguishing the flame. In addition, at low loads in a fired heater, carbon monoxide, nitrogen oxide, and nitrogen dioxide will increase.

Compared to a fired heater, very high fluid temperatures can be attained in electrical heaters. However, the coil metallurgy may still limit the design. Thus, ceramic tube coils may be used with electric heaters to attain a higher temperature. Further, single pass coils may be used, or other types of coils including multi-pass coils arranged in one row or in multiple rows. Since severity for each coil may be controlled independently, split cracking of different feeds through different coils may be simply achieved. Additionally, co-cracking of different feeds may be done by mixing the different feed streams and feeding the combined feed to the radiant coils.

After the feed in the coils 130 is heated to the reaction temperature, the reaction output may be directed from the reaction section 112 to a primary exchanger, such as a transfer line exchanger (TLE) 150 to be quickly cooled to an outlet temperature. When the reaction output is cooled in the primary TLE 150, a high pressure, high temperature steam may be generated. In some embodiments, the high pressure, high temperature steam may be directed to a preheating section of the reactor 100 to preheat feed prior to entering the reaction section 112. In some embodiments, the high temperature steam may be mixed with a feed and directed into the reaction section 112 to help with heating the feed for cracking.

Effluent from the primary TLE 150 may be directed to a secondary exchanger, e.g., TLE 160. In the secondary TLE 160, the effluent may be further cooled and steam generated. The steam generated from the secondary TLE 160 may be directed to the preheating section of the reactor and used to preheat the feed 105. In some embodiments, steam generated from the secondary TLE 160 may be directed into the reaction section 112 to help with heating the reaction section 112. In some embodiments, additional exchangers (e.g., a tertiary TLE or more) may be used in addition to a first and second exchanger (e.g., the primary TLE 150 and the secondary TLE 160).

In some embodiments, separate electric heating element(s) may be used with the primary TLE 150 and/or the secondary TLE 160 to superheat the steam generated by the TLE(s). By producing less steam in the TLE(s), additional heat in the effluent may be directed to preheating the reaction mixture. Therefore, maximum heat input to the reaction system 112 may go to cracking heat (e.g., more than 90% of the heat) and only a small amount to heating the steam (and a minimal amount of heat may be lost through the walls of the reaction section 112). In contrast, 10 to 40% of the fired heat in a fired heater may go to heating the steam and boiler feed water.

A preheating section of the reactor 100 may be integrally formed with the main reaction section in a single reactor unit, or a preheating section of the reactor may be provided separately from the main reaction section. According to embodiments of the present disclosure, all preheating of the feed to a reactor may be done by electricity. In some embodiments, common preheated and mixed feed with a dilution steam header can be employed. A preheating section may include one or more exchangers. In some embodiments, different feed types may be preheated in separate individual exchangers. For example, if the reactor 100 is to crack ethane, naphtha and gas oil, separate exchangers in a preheating section may be used to preheat each feed.

Since a common feed exchanger (e.g., TLE 150) may be used with the reactor 100 (e.g., which may receive different feeds through different coils in the reaction section), the cross over temperature (or inlet temperature to the reaction section) may be well controlled and nearly constant from the start of run (SOR) to the end of run (EOR). This is different from a fired heater. With coking in the radiant coils of a fired heater, the cross over temperature increases with time affecting the process performance. Therefore, usually a low cross over temperature is used at SOR so that metallurgical limits are not exceeded at EOR. Either feed/effluent exchangers and/or supplementary electrical heaters are used in conventional fired heaters to preheat the feed so that a constant temperature can be achieved at all times. With an electric heater, a high cross over temperature can be used from the beginning to reduce the electrical energy for the reaction section and reduce the cost of the heater (less number of radiant coils for a given ethylene capacity).

When only feed/effluent exchangers are used and no additional preheaters for the feed are installed, additional heat may also be supplied by the primary (reactor) electric heater 110. The heater 110 may be designed and configured to supply heat for preheating operations.

Examples of different possible parameters for a reactor according to embodiments of the present disclosure, such as shown in FIG. 1, is provided below merely to provide better understanding of embodiments disclosed herein. However, other parameters may be used while staying within the scope of this disclosure.

A first example of a reactor 100 may include:

Radiant coils having an inner diameter (ID) ranging from about 1 to 3 inches for the inlet tubes, an inner diameter ranging from about 2 to 4 inches for outlet tubes of a multi-pass coil, a length of between 20 and 50 ft, and containing between 100 and 200 tubes; and a linear TLE having an ID ranging from about 2 to 8 inches and length ranging from about 20 to 30 ft, and having between 40 and 50 tubes. For multi-pass coils, the inlet tube diameter and outlet tube diameters can be up to 8 inches or more and the total length can be up to 500 ft or more.

The first example reactor 100 may have the following operating conditions:

Naphtha Feed: S.G=0.703, P/N/A; COP (Coil Outlet Pressure): 30 psia; S/O=0.5; Feed rate=95026 lb/h at 8000 hours of operation; C2H4=29.0 wt %; C3H6=13.5 wt %; COT (Coil Outlet Temperature)=1596° F. (869° C.); and TLE outlet=1100° F. (593° C.).

Four radiant coil tubes may be combined to a linear TLE and quenched (e.g., as shown in FIG. 1). To preserve the yields the reaction has, the reaction output may be quenched quickly and the steam generation may be used. Saturated super high pressure (SHP) steam may be generated. By designing the TLE to provide conventionally low TLE outlet temperatures, the amount of duty for preheating the feed may be reduced. Therefore, instead of very low TLE outlet temperatures, a higher outlet temperature may be preferred (e.g., 1000-1200° F.). Even at higher outlet temperatures, the reaction may still be essentially quenched. The heat available in the effluent may still be high, but may not be sufficient to heat the feed to cross over conditions, which may also be relatively high (1000-1200° F.). For process optimization, TLE effluent may not be used for this service unless heater effluent is cooled to higher temperatures (e.g., greater than 1200° F.) (which may affect yield) or if the cross over temperature is set to lower temperatures (which may increase the radiant coil duty). In some embodiments, additional electric heater(s) may be used to preheat the feed to cross over temperatures without process optimization.

An arrangement of the reactor may include a radiant electric heater supplying the reaction heat followed by a TLE generating SHP saturated steam. The energy left in the effluents may be used to preheat the feed (e.g., naphtha feed) and/or dilution steam and/or mixed feed (e.g., naphtha+dilution steam) in shell and tube exchangers. To maintain a temperature approach, an additional electric heater may be used to preheat the feed to a cross over temperature. Instead of naphtha or hydrocarbon feed headers, other hydrocarbon (HC)+dilution steam (DS) mixed stream headers (hot) may be used. High temperature valves may be used to control the flowrates to a group of coils (or electric heater). Flow to individual tubes may be distributed via flow venturis (e.g., 124 shown in FIG. 1). Exchangers may be used for different feeds. For example, one exchanger for naphtha and one exchanger for gas feeds may be sufficient for an entire plant.

The effluents from an exchanger (e.g., secondary TLE 160) may be quenched further to about 200° C. with quench oil before entering a gasoline fractionator 170.

Operation Option-1

Low cross over temperature (˜1000° F.) with high TLE outlet temperature (˜1100° F.). When a secondary TLE 160 is used for heating the feed mixture (HC+DS), there may be at least 100° F. difference and a shell and tube exchanger design is possible. There may be an almost equal flow in tube side and shell side in the secondary TLE 160, and thus the temperature drop in the effluent side may be almost equal to the temperature gained in the shell side. The effluents can be cooled to 350° C. (662° F.). As such, a naphtha+DS feed mixture may be heated to 300° C. (572° F.) using only external means. A common feed preheater can be used instead of another electric preheater for each electric heater 110. By optimizing the primary TLE 150 outlet temperature, a separate electric preheater can be eliminated. Superheated dilution by other means may also be used to preheat a naphtha+DS mixture. The major heat load with a naphtha feed is the naphtha vaporization duty. When other sources like quench oil or low pressure or mid-pressure steam are used for vaporizing naphtha another electric heater can be avoided.

Operation Option-2

The reactor 100 may operate with a high cross over temperature and low TLE outlet temperature, and radiant duty may be the lowest compared with other operational options. A low TLE outlet temperature may be achieved in one stage (e.g., using the primary TLE 150) or two stages (e.g., using the primary and secondary TLEs 150, 160). In both stages SHP steam can be generated. In some embodiments, only the primary TLE 150 may be used for steam generation (for fast quenching). In some embodiments, the secondary TLE 160 may be used for preheating a HC+DS mixture (which may act similar to a lower mixed preheat (LMP) coil in fired heater convection sections, heating with effluents instead of flue gas).

Operation Option-3

Combinations of Operation Options 1 and 2 may be used with other additions. For example, a dilution steam may be superheated in a different electric heater and the superheated dilution steam may be used to preheat hydrocarbon (and partial steam) to cross over temperatures.

Though it is possible to carry out a cracking reaction in a single electric heater, heat balances may not work out for different feeds. When using a single electric heater for a cracking process, a portion of that electric heater may be dedicated to preheating the feed. Flow control may be based on high temperature streams, e.g., using valves 122 and flow venturis 124. Thus, temperature may be selected for improved reliability and cost effectiveness. Preheating may be a slow process and use more flowline surface area to be heated. Instead of using a separate electric heater for preheating, a shell and tube exchanger may be used for recovering the energy in the effluents to use in preheating. For example, feed may enter an electric heater at about 140° F. and the effluent may leave the reaction section at about 650° F. (before oil quench). With such temperatures, more than one electric heater may be used (when energy from other sources are not included) with a common feed preheater.

TABLE 1
Example calculations for Operation Option-1

| | |
|---|---|
| C2H4 capacity, KTA (8000 hours) | 100000 |
| HC Flow, lb/h/heater | 95026 |
| Ethylene Yield, wt % | 29.0 |
| Propylene Yield, wt % | 13.5 |
| HC (Nap)inlet at 140, enthalpy, BTU/h | 55.6 |
| Dil. steam at 392 F. enthalpy, BTU/h | 1221.3 |
| Cross over temperature (TXO), F. | 1000.0 |
| Coil Outlet Temperature (COT), F. | 1608.0 |
| S/O, w/w | 0.5 |
| Convec duty, MMBTU/h | 79.73 |
| Radiant duty | 131.15 |
| Total HC duty | 210.88 |
| Reaction Duty, MMBTU/h | 63.73 |
| TLE Outlet temp, F. | 1100.0 |
| SHP steam duty, MMBTU/h | 53.5 |
| TLE Effl. T before Tran. Line, F. | 660.0 |
| Addln Effl. duty, MMBTU/h | 41.67 |
| Total duty eff.(to660 F.) for preheat, MMBTU/h | 55.62 |
| Assumed availability, frac | 0.95 |
| Sec. TLE duty, MMBTU/h (for process heat) | 52.84 |
| min inlet T HC + DS to Sec. exchanger, F. | 417.04 |
| Convec still to be added by others, MMBTU/h | 26.89 |
| Naphtha duty, MMBTU/hr | 25.72 |
| direct Elect. heating efficiency, % | 90.000 |
| addln Convec duty requd, MMBTU/h | 26.894 |
| Convec Elec. Power, MW (sep. nap vap) | 8.764 |
| Radiant Elec. Power, MW | 42.734 |
| Total Elec. Power, MW | 51.498 |
| Thermodynamic Total Power in Heat, MMBTU/hr | 175.717 |
| Fuel equ. BTU/lb HC | 1849 |
| Elecy from nat. gas (40% ef)- Fuel equiv., BTU/lbHC | 4623 |
| Fuel. With convent. Fired heater, BTU/lb. HC | 2794 |
| Performance Improvement, % | 151.1 |

When electricity is produced from natural sources (e.g., solar or wind), and where the efficiency of production is irrelevant, then an electric heater may be 50% more efficient than conventional fired heaters. However, when electricity has to be produced with natural gas/fuel oil for the heat source, then electrical heating may not be economical.

Electrical Power Grid

Since a cracking process using electric heaters according to embodiments of the present disclosure may consume high amounts of electrical power, it may be advantageous to reduce electrical losses as much as possible. For example, when assuming electricity is available at site at high voltage with minimum loss from the generating station, there may still be limitations in equipment manufacturing with high voltage. Though most countries use 66 KV transmission line for long distance (e.g., from substation to substation), to the consumer 3000V to 11000V power may be available. In the ethylene industry, ID fan is a big electricity consumer. Most countries use 6000-6600V (e.g., PTTPE in Thailand, Petronas in Malaysia). For higher than 11 KV, corona discharge should be considered. Though the above calculation shows around 50 MW power may be a minimum amount of consumption, the following calculations are shown for 100 MW. A higher amount may be considered for higher capacity electric heaters or for multiple electric heaters.

TABLE 2
Copper electrical resistance and power loss

| Power, MW | 100 | 100 | 100 | 100 | 100 |
|---|---|---|---|---|---|
| Supply Volt | 250 | 440 | 3000 | 6000 | 11000 |
| Current (P.F = 1), amp | 400000 | 227273 | 33333 | 16667 | 9091 |
| Loss for 1 ohm, MW | 160000.0 | 51652.9 | 1111.1 | 277.8 | 82.6 |
| Loss for 0.01 ohm, MW | 1600.0 | 516.5 | 11.1 | 2.8 | 0.8 |
| Useful power ratio | 0.000 | 0.000 | 0.889 | 0.972 | 0.992 |
| Loss for 0.001 ohm, MW | 160.0 | 51.7 | 1.1 | 0.3 | 0.1 |
| Useful power ratio | 0.000 | 0.483 | 0.989 | 0.997 | 0.999 |
| Loss for .001 ohm | 545.9 | 176.2 | | | |
| MMBTU/h | | | 3.8 | 0.9 | 0.3 |

| | | Area | |
|---|---|---|---|
| Dia wire | Length | Cross sec | Resistance, |
| mm | M | M\hat{ }2 | Ohms |
| 1 | 1 | 7.854E−07 | 0.02190 |
| 10 | 1 | 7.854E−05 | 0.00022 |
| 10 | 10 | 7.854E−05 | 0.00219 |
| 10 | 50 | 7.854E−05 | 0.01095 |
| 15 | 50 | 0.0001767 | 0.00487 |
| 20 | 50 | 0.0003142 | 0.00274 |

Low voltages of about 250-440V may not be used without excessive power loss in the conductors (cable). The current requirement may be so high it is preferable to use 6000V and higher. The resistance may be extremely small, e.g., 0.001 ohm and lower, assuming a cable is 50 m from the transformer and 20 mm thick.

Control

Compared to a fired heater, electrical heating may be precisely controlled by regulating the power. Voltage regulators may be used to regulate the power. However, for high power cases, the power loss may be significant and may not be practical. In such cases, individual coil control may be preferred to overall electric heater control. That is, power to each coil (or a group of tubes) may be controlled. Also, by segmenting the power, temperature distribution may be maintained. For example, a 45 ft long coil can be segmented as 5 sections. Power to each section may be controlled (on or off), which may permit different severities in different coils, simultaneous cracking and decoking in different coils of the same heater, etc.

Other Aspects

Generally, in a conventional ethylene plant, liquid feed headers and gas headers are provided, where liquid feeds are vaporized. It is possible to find some low temperature heat sources that are available in the recovery section, e.g., naphtha+DS (0.2 w/w) feed. In this scenario, if an electric heater is used, one electric heater may be used for the whole plant. Similarly, a dilution steam may be superheated and fed to all electric heaters in the plant. Approaches like this may reduce the total number of electrical heaters that are required for cracking.

Though a single pass coil arrangement is considered in the above example, other types of coil arrangements may be used. Other coil arrangements may include multi-pass coil such as SRT-1 (a serpentine coil), SRT III (a four pass coil), SRT V, VI, or VII (two pass coils with multiple inlets and multiple outlets), U coil (one inlet with one outlet), Y coil (two inlets with one outlet) and other configurations. In contrast to conventional fired heaters where different types of heater coil designs may not be installed or operated within a radiant box, electric heaters according to embodiments of the present disclosure may include multiple different heater coil designs, including SRT-1 and SRT VI heater coil designs.

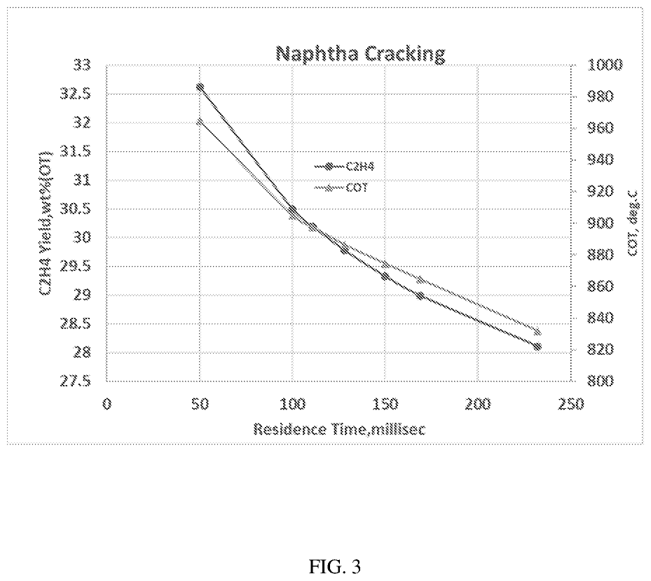

Coils may be made of ceramic material or a metallic material, including alloys such as carbon steel, austenitic stainless steel, Cr—Mo steel, other alloyed steels, and nickel-based alloys. When ceramic tubes are used, a relatively shorter residence time can be used (e.g., with metal tubes, higher than 950° C. gas temperature may be difficult). FIG. 3 shows a graph of the expected ethylene yield and COT as a function of residence time.

Since high temperature is possible using electric heaters of the present disclosure, steam only decoking may be used. Individual coils may also be decoked. To improve reliability, periodic decoking with steam/air may also be used.

According to embodiments of the present disclosure, a single header may be used to supply different feeds to electric heaters. Liquid headers (e.g., a naphtha header), gas headers (e.g., an ethane header), and/or mixed stream headers (e.g., a hot naphtha+dilution steam header or ethane+dilution steam header) may be used to supply a feed to one or more electric heaters. By using mixed stream headers, a maximum amount of the electrical energy may be used for feed preheating and a minimum amount of electrical energy may be used for steam generation.

In an electric heater, there may be many coils, which may be grouped in different groups or arranged together in a single reaction section of the electric heater. The coil outlet temperature may be controlled to optimize olefin production and to achieve a desired run length. Such control may be at least in part implemented by providing a group of coils with its own feed control valve. A single electric heater may have one group of coils or many groups. Unlike with fired heaters, an electrical heater may be divided into many subsections by placing insulations and/or diverting electrical energy to specific coils in a physical arrangement. The power consumption for an electric heater may be high (e.g., ranging from a few tens of megawatts to 100s of megawatts). Therefore, a power grid may be divided to supply each individual group of coils or to supply a few groups of coils. For controlling the temperature in groups of coils, a power grid may be segmented to supply power to each group of coils. In some embodiments, heating coils may be intertwined.

For example, with a three group system (e.g., 1-2-3; 1-2-3; 1-2-3) arranged vertically, when all 3 groups get the full power from the power grid, maximum heat may be released. When either group 1 or 2 or 3 is active, the power is ⅓^{rd} of the total power. When using a partial amount of the power, uniform heating throughout the coil may be maintained. The groups may be arranged vertically, where the bottom ⅓ or ½ can have a different power than the rest. In some embodiments, groups of coils may be arranged horizontally. Duty for full cracking to full decoking can be controlled precisely. Additionally, split cracking may be achieved with electric heaters. Two adjacent groups of coils may have different electrical power provided to each group.

Effluents in a reactor having an electric heater may be quickly cooled by generating steam. The effluent outlet temperature may be chosen to reduce steam production while also being able to quench the reaction. Excess energy in the effluent may be used to preheat a high temperature feed mixture (e.g., a mixed hydrocarbon and dilution steam feed), such that an additional heater to preheat the feed may not be needed. For a low temperature feed, already generated steam may be used. In such manner, a higher fraction of the supplied electrical energy may be used for the cracking process.

Methods

In cracking processes, a feed mixture may be heated to some temperature level (a reaction temperature) for the reaction to take place. In a conventional fired heater, energy in the flue gas may be used and additional energy may be used to generate high pressure steam. However, in an electrical heater, feed may be preheated by exchanging heat energy with the effluents from the reaction. A minimum amount of energy may be used with electric heater reactors to generate high pressure steam (which may be used in the recovery section or can be used to generate electricity back or preheat other process streams, when all compressors are electrically powered). Effluent from an electric heater may be quickly quenched to a level sufficient to slow the pyrolysis reactions. The quench/outlet temperature may be decided depending upon the type of feed. For example, when cracking ethane, the outlet temperature can be around 700 to 750° C. (e.g., where the reactor effluent is cooled to about 700° C. by generating steam). Further cooling of the effluents may be achieved by exchanging the heat with feed streams (e.g., ethane and dilution steam) in tubular exchangers. For naphtha crackers, the outlet temperature may range between 600-700° C., which is higher than outlet temperatures when using fired heaters.

In some embodiments, outlet temperatures may be lowered to generate more steam, which may be used in other areas of the reactor. For example, for ethane, an outlet temperature ranging from 350 to 450° C. may be selected to generate high pressure steam in a transfer-line exchangers (TLE) section of the reactor. For naphtha cracking, an outlet temperature ranging from 350 to 525° C. may be selected to generate steam. Relatively small transfer line exchangers (high pressure exchangers) may be used for high pressure steam generation for electric heaters according to embodiments of the present disclosure. When using relatively small TLEs, linear exchangers can be used and effluents can be combined for further cooling. Instead of linear exchangers, conventional exchangers can also be used.

Other exchangers (secondary and/or tertiary) may be used with electric heaters of the present disclosure, where feed may be exchanged with effluents using low pressure exchangers. In a fired heater, after generating steam with a primary exchanger, a secondary exchanger may be used for only some feeds (like ethane and propane, which have low fouling tendency). However, with electric heaters according to embodiments of the present disclosure, all feeds (gas and liquid feeds) may use secondary exchangers. These secondary exchangers may be installed with individual reactors to correspond with an electric heater or may be installed according to an overall plant design to correspond with each type of feed.

For example, a plant may have ethane and naphtha feeds directed to multiple conventional fired heaters, where for the purpose of example, 2 of the fired heaters can crack ethane, 5 of the fired heaters crack naphtha, and 1 spare fired heater can crack either one. In such an example, each ethane fired heater may have one secondary TLE, while the naphtha (and spare) fired heaters may not have any secondary TLE. When using electrical heaters according to embodiments of the present disclosure for the reactors in a comparison plant, all ethane electric heaters may be grouped, and ethane (optionally with a dilution steam) may be sent to one or more secondary exchangers that will heat the ethane (+dilution steam) feed for the ethane electric heaters. All naphtha electric heaters may be grouped and may exchange heat with the naphtha (and optionally mixed dilution steam) feed. The secondary exchangers can be arranged on an individual heater basis (e.g., many small exchangers) or feed basis (e.g., a few large exchangers for each feed type). In some embodiments, when designing on an individual heater basis, spare secondary exchangers may not be provided due to cost, while when designing on a feed basis, a spare secondary exchanger may be provided since a single spare secondary exchanger may service the entire plant.

Further, design simplification may be achieved using electric heaters disclosed herein, where after combining effluents from all primary TLEs (e.g., high temperature (greater than 600° C.) TLEs for ethane cracking heater(s) or naphtha cracking heater(s)) in a plant, the mixed effluents can be used to preheat the feed mixture. In this case, the total combined effluents can be divided into one or two or more streams. One effluent stream may go to preheating ethane and another effluent stream may go to preheat naphtha feeds. Secondary exchangers may also be designed to preheat both ethane and naphtha feeds independently in a single exchanger. Under these conditions, providing a spare secondary exchanger may not significantly increase the cost, but may increase the on-stream time significantly. Currently only primary TLEs can be cleaned on-line along with radiant coils in fired heaters, while secondary exchangers are cleaned mechanically (longer time and hence loss in production). By providing a spare secondary exchanger, on-stream time is increased (where effluent streams may continue to be directed where needed while other stream lines may be cleaned).

Electric heaters according to embodiments of the present disclosure may be used for different types of hydrocarbon cracking processes. For example, electric heaters disclosed herein may be used for thermal cracking processes for olefin production. Further, in addition to olefin production, electric heaters such as described herein may be used for catalytic reactors, e.g., for methane reformers or dehydrogenation reactors like propane dehydrogenation.

Different hydrocarbon feeds may be fed into electric heaters of the present disclosure for cracking. For example, hydrocarbon feeds may include C2, C3, C4, C5, . . . up to resids and whole crudes and any portion/fraction or mixture thereof, condensates and hydrocarbons with a wide boiling curve and end points higher than 500° C. Such hydrocarbon mixtures may include whole crudes, virgin crudes, hydroprocessed crudes, gas oils, vacuum gas oils, heating oils, jet fuels, diesels, kerosenes, gasolines, synthetic naphthas, raffinate reformates, Fischer-Tropsch liquids, Fischer-Tropsch gases, natural gasolines, distillates, virgin naphthas, natural gas condensates, atmospheric pipestill bottoms, vacuum pipestill streams including bottoms, wide boiling range naphtha to gas oil condensates, heavy non-virgin hydrocarbon streams from refineries, vacuum gas oils, heavy gas oils, atmospheric residuum, hydrocracker wax, and Fischer-Tropsch wax, among others. In some embodiments, the hydrocarbon mixture may include hydrocarbons boiling from the naphtha range or lighter to the vacuum gas oil range or heavier. If desired, these feeds may be pre-processed to remove a portion of the sulfur, nitrogen, metals, and Conradson Carbon upstream of processes disclosed herein.

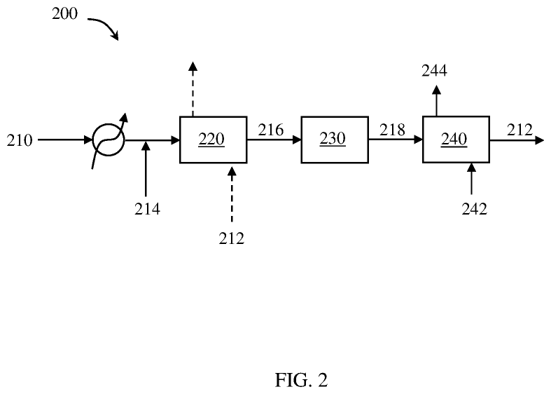

FIG. 2 shows a block flow diagram of a process 200 that may be used to thermally crack a hydrocarbon feed using an electric heater according to embodiments disclosed herein. As shown, a dilution stream 214, such as steam, may be added to the hydrocarbon feed 210 and preheated with effluents 212 in an exchanger 220. This can be done in one or more exchangers. Additional preheating may be done in a separate heater or combined with the main electric heater. Exchanger(s) and preheater(s) can be specifically designed for a single heater or may be generically designed to universally work in an entire plant. Further, exchangers and heaters may be designed to work together, which may be considered in the overall economics.

Once the feed mixture 216 is preheated to a desired inlet temperature, also known as the cross over temperature (TXO), the preheated feed mixture 216 may then enter the electric heater 230. The electric heater may superheat the feed mixture 216 to a reaction temperature, and the cracking reaction may proceed in coils of the electric heater 230 (e.g., in short residence time (SRT) coils). The flow to each coil may be distributed by control valves (e.g., high temperature valves) and venturis. The heat input into the electric heater 230 may be manipulated by adjusting the electrical input.

In the reaction section of the electric heater (in the coils), the process performance of the cracking may be the same as if the reaction took place in a conventional fired heater. In other words, no notable difference in the process performance may be detected in the reaction section of a conventional fired heater and an electric heater according to embodiments of the present disclosure. As such, electric heaters of the present disclosure may have a reaction section that provides the same or similar level of thermal cracking performance as a fired heater. In some embodiments, such as those providing uniform circumferential heating of individual coils, performance and selectivity may be improved, and may be due, in part, to a decrease in the number of or temperature of hot spots associated with fired heating.

Depending upon the electric heater design, the coil design can be modified. For example, a single pass design or multi-pass series-parallel arrangement may be used. In some embodiments, the coil design in an electric heater may be the same as the coil design in a fired heater (e.g., the same coil design as in the SRT® furnaces by Lummus Technology, including the SRT-I, SRT-II, SRT-III, SRT-V, SRT-VI, and SRT-VII fired heaters). In some embodiments, different coil arrangements may be used in a single electric heater. For example, a serpentine coil and a multi-pass split design coil may be arranged and operated in the reaction section of a single electric heater.

The severity of the thermal cracking may be adjusted by monitoring the outlet temperature of the output 218 from the electric heater 230 and adjusting the heat input into the electric heater 230. Further, the skin temperatures of the coils in an electric heater 230 may be measured and/or predicted using devices and methods used in fired heaters. For example, scanning infrared cameras, high-resolution imaging with focal plane array detectors, thermocouples, and selection of temperature measurement points may be used to monitor skin temperature of the electric heater coils, which may be used, for example, in determining the first stages of coking, corrosion, over- and under-balanced heat loads in the heaters, and prediction of the life of the coils.

The output 218 from the electric heater 230 may be directed to exchanger(s) (e.g., TLE(s)) 240, where it may be rapidly cooled down (quenched) after leaving the reaction section of the electric heater 230. Quenching the output 218 may be done to prevent secondary reactions and to stabilize the gas composition from the output. The same type TLEs used with conventional fired heaters may be used with electric heaters 230 of the present disclosure. For example, cooling of a cracked gas output 218 in the TLE 240 may be carried out by vaporization of high-pressure boiler feed water (BFW) 242, where the BFW 242 may be introduced around the TLE tubes to cool the cracked gas output 218 and vaporizes to generate high pressure steam 244. When cracking liquid feed (e.g., in processing heavy gas oil feeds), direct injection quench points may be provided to inhibit rapid fouling that may occur in the TLE cooling tubes when the cracked gas is cooled below the dew point of the heavy ends of the cracked gas.

Effluent 212 exiting the TLE 240 may be analyzed and directed to different paths for different uses, depending on the type of effluent. For example, effluent 212 may be heated and undergo additional cracking. In some embodiments, the effluent may be hydroprocessed to reduce a content of at least one of nitrogen, sulfur, metals, and Conradson Carbon in the hydrocarbon mixture. Same type equipment and processes as used with conventional fired heaters for combined effluent analysis may be used with electric heaters 230 according to embodiments of the present disclosure.

In some embodiments, effluents may be cooled to a relatively higher TLE outlet temperature when using electric heaters of the present disclosure compared with TLE outlet temperatures when using conventional fired heaters. For example, when using conventional fired heaters, effluents may be cooled to 350° C. to 400° C. (at start of the cracking cycle), generating high pressure steam (e.g., 115 bar) by cooling from a coil outlet temperature of 800 to 850° C. When minimum steam is required, such as when using electric heaters of the present disclosure, the TLE 240 outlet temperature can be increased to 600 to 650° C. At lower TLE outlet temperatures, thermal cracking reaction rates may be lower, which may use less energy to preheat the feed. Thus, lower TLE outlet temperatures may reduce the electrical consumption in electric heaters, but also reduces the steam production. An optimum outlet temperature/steam production may be determined for different thermal cracking processes. Examples of using different TLE outlet temperatures are considered below for illustration.

When minimum steam is considered, it is possible to eliminate the feed preheat and to heat the feed to a reaction temperature in a separate heater. Generally, the heater cross over temperature for naphtha is 1100 to 1175° F. (593 to 635° C.) and the heater cross over temperature for ethane is 1250 to 1300° F. (677 to 704° C.) in gas fired heaters. This level of preheating the feed cannot be achieved with effluent heating alone. By reducing the cross over temperature to 900° F. (482° C.) for naphtha and to 1000° F. (538° C.) for ethane, a separate electrical preheater may not be needed. Unfortunately, to reduce the cross over temperature, the tube skin temperature in the radiant coils may be increased and the run length reduced. For reasonable run lengths, more coils will be required. For liquid feeds it is difficult to eliminate the electrical preheater. Naphtha effluents may not be cooled below 350° C. or 300° C. since it will condense and foul the lines. However, ethane effluents may be cooled to 200° C., and that enthalpy may be used to preheat the ethane feed or the ethane/dilution steam mixed feed. This process may be done using secondary TLEs, where secondary TLEs may be used with conventional fired heaters or electric heaters. Further, conventional decoking and feed switching may be used with electric heaters according to embodiments of the present disclosure. For example, steam may be used to decoke coils in electric heaters disclosed herein.

Unlike conventional fired heaters, electric heaters according to embodiments of the present disclosure do not have a convection section. In electric heaters, a group of coils, e.g., 1 to 10 or 20 (or as many as practically feasible), may form the electric heater. The size of the coils and electric heater may be dictated by decoking capability.

One or more electric heaters may be used in an ethylene producing plant. Ethylene producing plants may have ethylene producing capacity well over 1800 KTA and an average ethylene producing capacity of greater than 1500 KTA of ethylene. To achieve such production, multiple electric heaters (e.g., six or seven operational electric heaters and a spare electric heater) may be used in the plant. Each electric heater in a plant may be designed to optimize ethylene production. For example, for a plant capable of producing 1000 KTA (kilotons per year) of ethylene, five groups of coils plus one spare group each forming an electric heater (where each group of coils/electric heater may have a 200 KTA size). As another example, a 2000 KTA plant may include five groups of coils plus one spare group each forming an electric heater (where each electric heater may have a 400 KTA size). A single electric heater may produce 200 KTA of ethylene or more, e.g., between 250 KTA and 300 KTA. In some embodiments, an electric heater producing 200 KTA of ethylene may have an electrical power consumption ranging from 65 MW to 130 MW. In some embodiments, an electric heater producing 1800 KTA of ethylene may consume as much as 1170 MW of total power.

Depending upon the electrical heating system (e.g., resistance, induction, and/or capacitance), heating may be supplied to each coil or each group of coils, and may depend on, for example, the electrical heater manufacturer. For example, in some embodiments, an electrical heater (e.g., an electric heater containing coils arranged as in SRT-VI® heaters) may have a common pipe for multiple feeds, e.g., Feed-1, Feed-2, etc. (e.g., Feed-1 may be naphtha, Feed-2 may be liquified petroleum gas (LPG), Feed-3 may be ethane, etc.). The feed(s) may be preheated outside the electrical heater where the pyrolysis reaction is carried out. A group of coils and the TLE (generating steam) may form an electric heater according to embodiments of the present disclosure and may be isolated for decoking or repair.

In fired heaters, large capacity heaters may use a twin cell radiant box design, where a twin cell radiant box design may include two radiant cells in a common convection section. Single cell design fired heaters can be used to build 200 KTA capacity. Since electric heaters do not have convection sections as in conventional fired heaters, a 200 KTA ethylene production may be used as a basis for comparison of an electric heater with a fired heater. However, ethylene production from an electric heater may be less or more than 200 KTA (e.g., ranging from about 170 KTA to 400 KTA or more). Examples of electric heater designs are provided herein for naphtha and ethane cracking based on 200 KTA of ethylene. For simplicity, full range naphtha at high severity and pure ethane at 65% conversion have been considered. Further, while different coil arrangements may be used in electric heaters (e.g., the coil arrangements used in SRT-I, SRT-II, SRT-III, SRT-V, SRT-VI, or SRT-VII by Lummus Technology or single pass coil arrangements), examples of electric heater designs are presented using a coil arrangement matching the coil arrangement in the SRT-VI fired heater by Lummus Technology, which is a high selective two-pass coil with long run length. Electric heaters with this coil arrangement can be used for both naphtha and ethane cracking to produce ethylene. Standard coil outlet pressure of 25 psia may be used. A steam to oil ratio (S/O) of 0.1 to 1.5 w/w may be used for various feeds; for example, 0.5 w/w may be used for naphtha cracking and 0.3 w/w for ethane cracking. An electric heater may run for at least 45 days.

Naphtha properties include a specific gravity (SG) of 0.707, initial boiling point (IBP) of 91° F. (33° C.), 50v % of 189, final boiling point (FBP) of 348° F. (176° C.), 74.6 wt % paraffins, 16.65 wt % naphthenes, 8.75 wt % aromatics, and an interference-to-noise power ratio (UN Ratio of P) of 0.83. 100% pure ethane may be used for thermally cracking ethane in an electric heater.

Table 3, provided below, gives example design and operating parameters for electric heaters capable of thermally cracking naphtha and ethane to produce ethylene. Case 1 corresponds to a naphtha heater design and Case 2 corresponds to an ethane heater design.

TABLE 3
Electric Heater Design for Ethylene Production

| CASE No | 1A | 1B | 2A | 2B |
|---|---|---|---|---|
| Ethylene capacity, KTA | 200 | 200 | 200 | 200 |
| Operating hours | 8000 | 8000 | 8000 | 8000 |
| Coil Type | SRT VI | SRT VI | SRT VI | SRT VI |
| Feed | Naphtha | Naphtha | Ethane | Ethane |
| Purity, % | 100 | 100 | 100 | 100 |
| Severity/Conv | High | High | 65 | 65 |
| C2H4, wt %(OT) | 30.70 | 30.70 | 52.6 | 52.6 |
| C3H6, wt %(OT) | 14.20 | 14.20 | 0.8722 | 0.8722 |
| Feed Rate, kg/h | 81433 | 81433 | 47529 | 47529 |
| Steam to oil ratio (S/O), w/w | 0.5 | 0.5 | 0.3 | 0.3 |
| COP, psia | 25 | 25 | 25 | 25 |
| # Coils/heater | 8 | 9 | 8 | 9 |
| Configuration | 24/4 | 24/4 | 20/4 | 20/4 |
| ID in/out, in | 2/5 | 2/5 | 1.8/4 | 1.8/4 |
| Length, ft/pass | 45 | 45 | 46 | 46 |
| As electrical Heater | | | | |
| Rad. duty, MMBTU/h | 216.5 | 236.6 | 161.6 | 172.9 |
| Elect. Efficiency, % | 90.0 | 90.0 | 90.0 | 90.0 |
| Power required for Reactor, MW | 70.5 | 77.0 | 52.6 | 56.3 |
| Coil Outlet Temp., F. | 1557.9 | 1552.4 | 1566.6 | 1560.0 |
| Primary TLE outlet Temperature, F. | 680.0 | 1202.0 | 680.0 | 1202.0 |
| TLE duty, MMBTU/h | 176.9 | 71.1 | 100.3 | 45.6 |
| SHP steam (sat), T/h | 87.9 | 35.3 | 49.8 | 22.7 |
| Heater Effluent Out Temp., F. | 660.0 | 660.0 | 400.0 | 400.0 |
| HC + DS Duty BL to TXO, MM BTU/h | 182.3 | 162.5 | 104.7 | 93.2 |
| Total Process duty, MMBTU/h | 398.8 | 399.1 | 266.2 | 266.1 |
| Duty used for preheating, MMBTU/h | 0.0 | 77.1 | 24.7 | 68.6 |
| Electrical duty-Process, MMBTU/h | 398.8 | 322.0 | 241.5 | 197.5 |
| Electrical Power with eff., MW | 129.9 | 104.8 | 78.6 | 64.3 |
| % Power for Radiant section | 54.3 | 73.5 | 66.9 | 87.5 |
| Power KW/T of ethylene | 5195.0 | 4193.9 | 3145.6 | 2572.5 |

Cases 1A and 2A correspond to conditions with high cross over temperatures and low TLE outlet temperature (to maximize steam production), where all the duty may be supplied by the electric heater(s). This produces a maximum amount of steam. Cases 1B and 2B produce lower amounts of steam. The heat available in the effluent may be used to preheat the feed to a maximum extent. For some feeds, maximum preheating of the reaction mixture to the cross over temperature is possible without the use of a separate electrical heater. In some embodiments, a separate electrical heater may be used for super heating steam (to about 500° C.).

With high cross over temperature (or preheat temperature), radiant coil surface area may be reduced, which may allow the electric heater to run for at least 45 days. For example, as shown in Cases 1A and 2A, an electric heater with 8 coils arranged in an SRT-VI configuration may achieve a 200 KTA capacity. With a lower cross over temperature, more coils may be used to achieve the same capacity. For example, in Cases 1A and 2A, 8 coils are used, and in Cases 1B and 2B, 9 coils are used to achieve the same capacity. When more coils are added, a still lower cross over temperature can be used without using a separate electrical heater for any feed.

For ethane cracking, since the hydrocarbon feed rate is low (due to high ethylene yield), the heat transfer coefficient is low. To get a maximum benefit, a slightly different SRT-VI design may be considered for ethane cracking. However, whatever coil design is considered for the A cases, the B cases may be similarly designed with one more coil than the A cases.

Table 4, provided below, gives another example of design and operating parameters for electric heaters capable of thermally cracking naphtha and ethane.

TABLE 4
Additional Examples for Electric Heater Design

| Feed | Naphtha | Ethane |
|---|---|---|
| Flow, kg/h | 95434 | 57745 |
| S/O | 0.50 | 0.30 |
| COP, bara | 1.72 | 1.72 |
| Cross over Temp., C. | 608 | 707 |
| Coil Outlet Temp., C. | 828 | 858 |
| TLE Outlet Temp., C. | 356 | 347 |
| Bulk Res. Time, sec | 0.23 | 0.25 |
| C2H4 Production, KTA | 200 | 251 |
| Total Heat Tr. area, m2 | 823 | 869 |
| Heat Flux, Kcal/m2 · hr | 74 | 58 |
| Box inside Length, m | 31.7 | 34.8 |
| Average Heat Flux, KW/M2 (OD Basis) | 86 | 68 |
| Radiant Duty, MW | 71 | 59 |

A naphtha heater may utilize more power than that of an ethane cracking heater. For example, the reaction section alone in a naphtha heater may have a minimum power consumption of about 70 MW/heater, whereas the reaction section of an ethane heater may have a minimum power consumption of about 52 MW/heater. When preheating is performed prior to cracking, the total power used may be 10 to 20% more than the power consumption of the reaction section alone. For this calculation, a 90% efficiency may be assumed for electrical heaters, but a more than 95% efficiency may be possible. For example, in electrical heating, 90 to 98% of the electrical energy may be used for the reaction. Thus, there may be little to no recovery of heat that was not used for the reaction. Because only an amount of energy just sufficient for the reaction may be supplied by the electric heater, there is substantially no excess or wasted energy use.

Further, since there is no convection section and burners in electric heaters of the present disclosure, electric heaters of the present disclosure may be arranged differently from a conventional fired heater layout. Accordingly, the plot space of reactors using electric heaters of the present disclosure may be reduced when compared with fired heaters.

An electric heater may have an electrical power requirement ranging from 2600 KW to 5200 KW per ton of ethylene. When producing 1800 KTA of ethylene, an electric heater may use about 580 MW to thermally crack ethane, and as much as 1170 MW when thermally cracking naphtha. Additional energy may be used for superheating steam used in the cracking process and for the recovery section. For example, about 600 MW of power may be used for an ethane cracker and about 1300 MW of power may be used for a naphtha cracker for the whole plant (including the electric heater, preheating, and recovery components). An energy source used to power an electric heater (and/or supporting components for preheating and recovery) may be, for example, nuclear power, hydraulic, solar, wind, or renewable methods. In some embodiments, a fossil fuel may be used to generate electricity for the electric heater plant. However, use of a fossil fuel for electricity generation may counter the environmental benefits for using an electric heater. Additionally, when excess electricity is used in an electric heater or elsewhere, the resulting excess heat energy may be converted back to electricity (e.g., using generators).

The specific energy of an electric heater when thermally cracking naphtha to produce ethylene may be about 5700 KW/T (kilowatt/ton) of ethylene or less, and when thermally cracking ethane to produce ethylene may be about 4200 KW/T of ethylene or less. When steam is not generated in the heaters, additional energy may be needed to electrically power the recovery section. Thus, according to embodiments of the present disclosure, electricity usage throughout an entire plant, including preheating component(s), electric heater(s), and recovery component(s), may be pre-planned to account for different thermal cracking processes that may be used in the plant and/or different feeds that may be thermally cracked.

According to embodiments of the present disclosure, plant design may also include consideration of start-up conditions. Additionally, planning may also include consideration of generation and consumption of steam that occurs from thermal cracking, e.g., determining what steam levels should be generated to reduce total energy consumption to below a certain amount, and generation of dilution steam from heat exchange with the process streams. For example, with complete electrification of a plant, outside steam can be reduced to a minimum, and start-up boilers may possibly be eliminated when the plant is configured properly. Complete steam balance may be determined before deciding on the electrical power amount for the electric heater. For example, dilution steam can be superheated so that the energy balance of the cracking heater does not affect the cracking severity significantly. The dilution steam may be superheated in the same heater where the feed is cracked, or the dilution steam can be superheated in separate heaters. Selection of an integral or separate dilution steam super heater may depend on the energy available.

Methods for designing a thermal cracking plant (including an electric heater for thermally cracking a feed, a recovery section, and optionally, a preheating section) may include determining an amount of steam generated and an amount of steam consumed by the thermal cracking plant, determining an amount of power used by the thermal cracking plant to thermally crack the feed, and adjusting at least one parameter of the thermal cracking plant to reduce the amount of power used by the thermal cracking plant. Parameters that may be adjusted to alter the amount of energy used by the thermal cracking plant may be selected from at least one of lowering a cross over temperature of the feed to the electric heater, designing the electric heater to have at least one additional coil to lower the cross over temperature of the feed, increasing an outlet temperature from the recovery section, reducing the amount of steam consumed by the recovery section, increasing the amount of steam consumed by the preheating section, and others discussed above.

Using electric heaters for thermal cracking may require more power for the thermal cracking than when electric heaters are used in other industries (e.g., for iron ore melting). For example, while electric heaters in other industries may have a maximum power consumption in the order of a few kilowatts, the power consumption of electric heaters disclosed herein used to crack a hydrocarbon feed may be in the order of many megawatts. Thus, methods of the present disclosure may include designing the thermal cracking plant to use the least amount of power while still being capable of thermally cracking a selected feed. In some embodiments, electric heaters may be modularized, which may allow for design adjustments depending on the thermal cracking process and feed. Other separation techniques like adsorption/absorption may be considered when designing the plant. When alternates to cryogenic separation is available, small scale chemical grade olefins can be very attractive with this route.

In contrast to a fired heater, an electric heater may maintain a constant cross over temperature throughout the thermal cracking process run. Additionally, unlike a fired heater, an electric heater may maintain a constant cross over temperature for low to high severities and low to high throughputs.

Further, electric heaters of the present disclosure do not generate flue gas, and thus may include only radiant section(s) and effluent cooling section(s). Thus, efficiency of electrical heating may be much superior to that of fired heating, where typically 35 to 45% of radiant duty is absorbed in gaseous fuel heating. By controlling the heat loss (where electric heaters don't have radiant duty absorbed in gaseous fuel heating), more than 95% of the electrical energy used to generate heat may be absorbed in the process. Therefore, the reaction section duty in an electric heater may be relatively small compared with a fired heater. However, the flue gas generated in a fired heater may be used to preheat the reaction mixture to the required reaction inlet temperature (cross over temperature, TXO), whereas electric heaters do not have flue gas for preheating. Overall fuel efficiency (thermal efficiency) including the preheating flue gas may be about 94%. When using fired heaters, even if heating the reaction mixture from battery limit conditions to reaction conditions, additional energy is available in the flue gas. Flue gas may be used to generate and superheat high pressure steam, which may be used in the recovery section for driving the compressors. Though the radiant efficiency is low, thermodynamic utilization of fuel energy is much higher.

Since there is no flue gas with electrical heating, most of the heat used in the process may go to the reaction. Thus, the amount of steam generated during the process may be reduced significantly. Steam generation may be used in cracking processes as a way to recycle heat (e.g., to preheat feed prior to entering the reaction section of the heater), and thus, when steam generation is reduced, other heating options may be used to compensate for the reduced amount of steam. For example, additional preheating of feed may be carried out with a second electric heater. When an entire thermal cracking plant (e.g., including one or more main reaction heaters, one or more recovery sections (e.g., exchangers), one or more preheating sections (e.g., a preheating heater), and/or post processing equipment) is using electrical energy, optimization of preheating and reaction heating can be done in a more efficient way. For example, heat generated from one plant equipment unit (e.g., from a main reaction electric heater) may be recycled to another plant equipment unit (e.g., to a preheating section). Optimization of preheating may also be conducted for a single electric heater, where thermal energy (high temperature) from reactor effluent may used to preheat the feed and/or to generate steam.

Currently, ethane crackers may produce significant amount of steam compared with the feed rate (— 2 kg SHP superheated steam/Kg of ethane feed). Ethane heaters also use some sort of preheating (secondary TLEs). For gas cracking using electric heaters, electrical demand can be reduced by preheating the feed with the effluent as much as possible. Some level of external reaction mixture preheating may be conducted when using electric heaters, which may be done by additional electrical heating. Under certain circumstances this can be included in the main reaction heater or a separate preheater. The size and/or costs of electric heaters may be considered as a function of electrical demand to optimize design of sourcing preheater energy (e.g., from a main reaction heater or separate preheater).

With electrical heating, the heating rate may be uniform, and the inputted heat flux can be adjusted by manipulating the electrical input. A maximum metal temperature may occur proximate to the end of the coil. With some heater designs there is no shadow factor. Hence, expected maximum tube metal temperature (TMT) can be considerably lower in electric heaters than that observed with fired heaters. This can reduce the cost of the electric heater. Other benefits of using an electric heater may include, for example, control philosophy, plot space modularization, etc.

As described herein, electric heaters may offer advantages over conventional fired heaters. For example, in an electric heater, only the duty required for the reaction may be supplied while taking into account only minor loses (whereas in fired heaters, much of the fired duty may be lost in flue gas). Further, the reactor effluents from an electric heater may be used to preheat the feed, thereby reducing the total duty supplied to the reactor. Electric heaters may also be more compact when compared with fired heaters (which include both a radiant section and a convection section).

Additionally, electric heaters may provide more controlled heating than fired heaters. For example, electrical heating may be more uniform than heating from a fired heater, and the heating rate can be controlled better in an electric heater when compared to a fired heater. Further, selected coils in an electric heater may be selectively controlled (e.g., controlled heating of a single coil or groups of coils), such that olefins can be produced much more selectively.

Safety may also be improved using electric heaters. Most heater accidents happen during start-up and shut down, often due to improper handling of fuel safety standards. Since no fuel may be used with electric heaters, fuel-type safety incidents may be eliminated or reduced. Additionally, because the structure of electric heaters according to embodiments disclosed herein may be simplified when compared with convention heaters, safety may not be as much of concern in high earthquake regions and at high wind loads (e.g., due to a low structure height and no fuel use).

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed:

1. A reactor for cracking a hydrocarbon feed, comprising:
a heater chamber defining a reaction section of the heater;
a plurality of electrical heating elements disposed within the heater chamber, wherein the electrical heating elements are electrically powered;
at least one coil extending from a feed inlet through the reaction section; and
a primary exchanger comprising an inlet fluidly connected to the at least one coil and an effluent outlet.

2. The reactor of claim 1, further comprising a secondary exchanger having an inlet fluidly connected to the effluent outlet of the primary exchanger.

3. The reactor of claim 1, wherein the primary exchanger further comprises a steam outlet and a steam flow line directed to the feed inlet.

4. The reactor of claim 1, further comprising a preheating section spaced apart from the reaction section and downstream of the feed inlet, wherein the preheating section comprises at least one exchanger.

5. The reactor of claim 1, wherein the feed inlet is fluidly connected to multiple feed sources.

6. A method of thermally cracking a hydrocarbon feed, comprising:
feeding the hydrocarbon feed into at least one coil in a reaction section of an electric heater;
using electrical energy to heat the hydrocarbon feed in the electric heater to a reaction temperature; and
directing a reaction output from the electric heater to at least one exchanger to cool the reaction output.

7. The method of claim 6, further comprising recovering heat from the reaction output using the at least one exchanger and using the recovered heat to preheat the hydrocarbon feed prior to feeding the hydrocarbon feed into the electric heater.

8. The method of claim 6, further comprising selectively heating different sections of the at least one coil to selected temperatures using multiple electric heating elements disposed around the at least one coil in the electric heater.

9. The method of claim 6, further comprising feeding multiple different types of feeds to different coils in the reaction section and collectively separating the reaction output from the multiple feeds.

10. The method of claim 6, further comprising feeding a second hydrocarbon feed into the electric heater, the second hydrocarbon feed having a different composition than the hydrocarbon feed.

11. The method of claim 6, further comprising:
using a valve to isolate one of the at least one coil; and
decoking the isolated coil.

12. A method, comprising:
designing a thermal cracking plant comprising:
an electric heater for thermally cracking a feed; and
a recovery section;
determining an amount of steam generated and an amount of steam consumed by the thermal cracking plant;
determining an amount of power used by the thermal cracking plant to thermally crack the feed; and
adjusting at least one parameter of the thermal cracking plant to reduce the amount of power used by the thermal cracking plant.

13. The method of claim 12, wherein adjusting the at least one parameter comprises lowering a cross over temperature of the feed to the electric heater.

14. The method of claim 13, further comprising designing the electric heater to have at least one additional coil to lower the cross over temperature of the feed.

15. The method of claim 12, wherein the thermal cracking plant further comprises a preheating section, and wherein adjusting the at least one parameter comprises increasing an outlet temperature from the recovery section, reducing the amount of steam consumed by the recovery section, and increasing the amount of steam consumed by the preheating section.
